(19) United States
(12) Patent Application Publication
(10) Pub. No.: US 2018/0177915 A1
(43) Pub. Date: Jun. 28, 2018

(54) METHOD FOR PREPARING TRANSPARENT COLLAGEN MATRICES

(71) Applicants: UNIVERSITE PIERRE ET MARIE CURIE (PARIS 6), Paris (FR); CENTRE NATIONAL DE LA RECHERCHE SCIENTIFIQUE, Paris Cedex 16 (FR); ECOLE PRATIQUE DES HAUTES ETUDES, Paris (FR); COLLEGE DE FRANCE, Paris Cedex 05 (FR)

(72) Inventors: Nadine NASSIF, PARIS (FR); Marie-Madeleine GIRAUD-GUILLE, PARIS (FR); Yan WANG, Warterloo (CA); Marc ROBIN, Chatillon (FR); Flore SALVIAT, Paris (FR)

(21) Appl. No.: 15/735,992

(22) PCT Filed: Jun. 17, 2016
(86) PCT No.: PCT/FR2016/051492

§ 371 Date: Dec. 13, 2017

(30) Foreign Application Priority Data
Jun. 24, 2015 (FR) 15/55832

(51) Int. Cl. A61L 27/24; A61L 27/50

(52) U.S. Cl. CPC A61L 27/24; A61L 2430/16; A61L 27/50

(57) ABSTRACT

Disclosed is a method for preparing a transparent fibrillated collagen matrix capable of being used as a tissue substitute in the production of a tissue or an artificial organ, in particular for a cornea substitute.

Collagen is a family of proteins, widespread in the animal kingdom, that represents approximately 30% of vertebrate proteins and is also found in certain invertebrates. Collagen is also ubiquitous in a significant number of tissues of one and the same organism where it contributes to the structuring of extracellular matrices and provides, in addition to a mechanical protection, anchor points to the surrounding cells. Collagens that are organized in fibrils during a process called fibrillogenesis are those most frequently found in tissues and represent ˜90% of all collagens. In physiological conditions, fibrils form ordered three-dimensional structures.

In living tissues, several types of biological macromolecular organization are found depending on the mechanical properties required (Hulmes D. J. S., Miller A. 1979. Nature, 282, p. 878-880.). For example, a cholesteric phase structure is observed in compact bone and the bone trabeculae of spongy bone; this three-dimensional organization of collagen contributes to the mechanical properties of the bone. It can be recreated and stabilized in vitro. Similarly, it is well known that the organization of collagen fibrils is unidirectional in the tendons while it has the form of plywood locally in the cornea.

The cornea is the first retractive element of the eye; it covers approximately one-fifth of the surface of the eyeball. Diseases which opacify the cornea are a major cause of blindness. A cornea transplant from a healthy cornea taken from a deceased donor can cure this, but the failure rate is quite high due to rejection of foreign tissues by the receiving organism.

Cornea substitutes are used but these are generally based on synthetic polymers.

Techniques making it possible to obtain transparent fibrillated collagen matrices have been described.

Application WO2012/52679 claims a particular method of fibrillogenesis and the description teaches that the concentration of the collagen solution can be comprised between 10 and 200 mg/mL, advantageously between 10 and 90 mg/mL and more advantageously between 30 and 80 mg/mL. The method is described as being able to be used in order to prepare a transparent matrix of fibrillated collagen capable of being used as a biomaterial, in particular as a corneal substitute. In Example 1, a collagen acid solution is concentrated by centrifugation to a final concentration comprised between 27 and 45 mg/mL which is then dialysed against PEG in order to obtain the formation of fibrils. A maturation period from a few days to 2 months is necessary in order to obtain a solid and stable matrix.

Application WO2011/151587 describes a particular method for preparing homogeneous material based on collagen by concentrating a collagen solution, comprising a dialysis step. Various possible values are shown for the concentration of the collagen solution: 5, 10, 20, 30, 40 et 250 mg/mL. In Example 1, the preparation of matrices with a concentration of approximately 250 mg/ml are described. In Example 2, intramuscular implantation is described of collagen matrices at 20 mg/mL and 40 mg/mL, the preparation of which is not described. In both cases, the matrices obtained are not transparent.

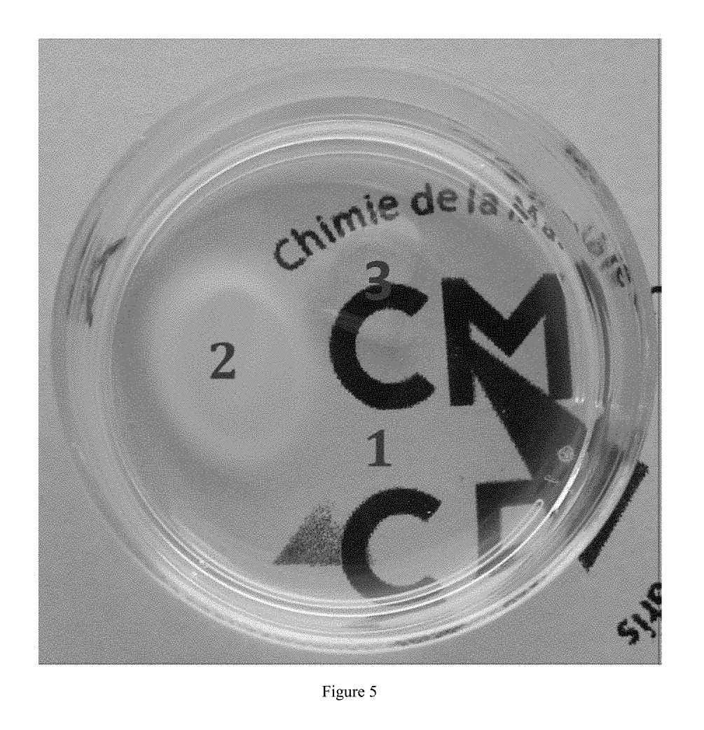

Application WO2015/049646 describes a method in which the collagen acid solution comprises a weak acid and a strong acid and in which the concentration is between 10 and 250 mg/mL, preferentially between 30 and 200 mg/mL and advantageously between 40 and 120 mg/mL. According to FIG. 23, the 45 mg/mL concentration was not carried out and according to FIG. 12, the preferred concentration values appear to be between 90 and 120 mg/mL.

There is also a need to develop a simple and rapid method that makes it possible to quickly have available a transparent matrix of fibrillated collagen having the structure closest to the cornea which remains transparent over a long period and which does not require a long maturation period in order to be transparent and stable.

In the course of their work on collagen solutions, the inventors have developed a method for preparing fibrillated collagen matrices making it possible to obtain a structure close to that of the cornea without the need for storage time.

Therefore, a subject of the present invention is a method for preparing a transparent matrix of fibrillated collagen comprising the following steps:
a) a step of preparing a collagen acid solution in an aqueous solvent
b) a step of concentrating the collagen acid solution
c) a step of fibrillogenesis of the collagen matrix
characterized in that the concentration step is carried out until a collagen concentration comprised between 43 and 50 mg/ml is obtained.

In a particular embodiment, the subject of the present invention is a method for preparing a transparent matrix of fibrillated collagen comprising the following steps:
a) a step of preparing a collagen acid solution in an aqueous solvent
b) a step of concentrating the collagen acid solution
c) a step of fibrillogenesis of the collagen matrix
characterized in that the concentration step is carried out
i) so as to limit, advantageously to avoid, shearing stresses, and
ii) until a collagen concentration comprised between 43 and 50 mg/mL is obtained.

Without wanting to be bound to any particular theory, the inventors think that this property of transparency observed in this interval is associated with an organization of the collagen in a particular liquid-crystal form that may be similar to a “blue phase”, i.e. an intermediate liquid crystal phase between an isotrope phase and a cholesteric phase.

According to the invention, the collagen can be of natural, recombinant or synthetic origin. Preferentially, the collagen is a collagen of type I, II, III, IV and/or V. Advantageously, the collagen is of type I, III or V, alone or in a mixture.

According to the invention, the concentration step can be carried out by evaporation or by dialysis.

In a particular first embodiment, the concentration is carried out by evaporation. This technique for concentrating collagen, well known to a person skilled in the art, consists of evaporating a solution of diluted collagen of a known volume (or mass) and concentration under sterile conditions, for example under a laminar flow hood. The solution is left to evaporate to the desired concentration (Hélary et al. 2005. Biomaterials, 26, p. 1533-1543). This technique makes it possible to estimate fairly precisely the concentration during evaporation by simple weighing.

In a particular second embodiment, the concentration is carried out by dialysis. In this technique, the collagen acid solutions, the initial concentration of which is generally less than or equal to 5 mg/mL, are brought into contact with a dialysis membrane. In these particular embodiments, the collagen solutions to be concentrated can be placed in dialysis tubing (Knight et al. 1998. J Biomed Mater Res. 41, p. 185-191.) or injected in a controlled manner into a dialysis cell as described in the patent document WO2011/151587. In both cases, the assembly is brought into contact with an osmotically active polymer solution the molecular weight of which is greater than the pore size of the dialysis membrane. A person skilled in the art can easily determine the dialysis parameters (type of membrane, concentrations of solutes of counter-dialysis liquids) based in particular on the laws governing the phenomena of transport through membranes. The osmotically active polymer Dextran® and polyethylene glycol (PEG) can be cited by way of example. Advantageously, the polymer is polyethylene glycol the molecular weight of which is 35,000 Da.

Within the meaning of the present invention, the concentration step is carried out so as to limit, advantageously to avoid, shearing stresses.

In an advantageous embodiment of the invention, the concentration step is carried out until a collagen concentration is obtained comprised between 43.5 and 47 mg/mL, preferentially of the order of 45 mg/mL. Within the meaning of the present invention, “of the order of 45” means for example 45±2 mg/mL, preferentially 45±1 mg/mL.

According to the invention, the collagen acid solution can be prepared according to techniques known to a person skilled in the art, in particular the technique described by Gobeaux et al. (Langmuir, 2007, 23, 6411-6417). In these solutions, the collagen is only in the form of monomers and optionally in the form of aggregates.

In a preferred embodiment of the invention, the collagen acid solution is constituted by an aqueous acetic acid solution. In a particular embodiment of the invention, the collagen acid solution is constituted by an aqueous acetic acid solution in the absence of a strong acid.

The method according to the invention is advantageously implemented when the concentration of the initial acid solution, before the concentration step, is comprised between 0.01 mg/mL and 5 mg/mL. When concentration is carried out by evaporation, a collagen acid solution of a concentration comprised between 3 and 5 mg/ml is preferred. When concentration is carried out by dialysis, a collagen acid solution of a concentration comprised between 0.5 and 3 mg/ml is preferred. According to the invention, the formation of the fibrils, also called the fibrillogenesis step, consists of bringing the collagen matrix into contact with a basic gas phase or a neutral or basic liquid. In the case where concentration is carried out by dialysis, the fibrillogenesis step can take place either in situ by replacing the osmotically active polymer with the gas or liquid phase, or by immersing the collagen matrix in a gas or liquid phase. Advantageously, the fibrillogenesis step is carried out so as to limit, advantageously practically to avoid, shearing stresses. According to a particular embodiment of the invention, the fibrillogenesis step is carried out by bringing the collagen matrix into contact with ammonia vapours.

Optionally, the method according to the present invention also comprises a step of washing the fibrillated collagen matrix in a buffer solution, for example phosphate buffered solution.

A subject of the present invention is also a method for preparing a transparent collagen matrix comprising the following steps:
a) preparing a collagen acid solution in an aqueous acetic acid solution at a collagen concentration comprised between 0.1 and 5 mg/mL, advantageously between 0.2 and 1 mg/mL;
b) concentrating the collagen acid solution by evaporation or by dialysis until a collagen concentration is obtained comprised between 42 and 50 mg/mL, preferably comprised between 43 and 47 mg/mL, more preferentially of the order of 45 mg/mL;
c) forming fibrils by bringing the collagen matrix into contact with a basic gas phase or a neutral or basic liquid, advantageously by bringing the collagen matrix into contact with ammonia vapours.

A subject of the invention is also a transparent fibrillated collagen matrix capable of being obtained by a method according to the invention, characterized by a transmittance greater than 0.6, preferably greater than 0.8 at 700 nm.

According to an advantageous embodiment of the invention, the matrix has blue phases, said blue phases being observed by PLM.

In an embodiment of the invention, the matrix has a pH greater than or equal to 7.

Within the meaning of the present invention, by “transparent matrix” is meant a matrix the UV-visible absorption density of which is equivalent to that of a non-fibrillated concentrated collagen solution.

Advantageously, the matrix according to the invention is stable. Within the meaning of the present invention, by “stable transparent matrix of fibrillated collagen” is meant a matrix that does not exhibit substantial changes in its transparency, preferentially of its transmittance at 700 nm, over several months. Such matrices have for example been stored for 4 years at 4° C. in sterile ultrapure water without alterations in transparency.

A subject of the present invention is also the use of a transparent fibrillated collagen matrix thus obtained as a tissue substitute for the production of a tissue or an artificial organ, in particular of a cornea substitute. This collagen matrix can also be used for the production of a dressing.

### EXAMPLES

The solutions used in the examples are described hereinafter:

Acetic acid (CH3COOH 500 mM): 58 mL Purex glacial acetic acid (Carlo Erba®, Purity>=99.8%) is diluted in 2L final volume of distilled water. The solution is then sterilized using a filter unit (Nalgene®, polyethersulphone, pore size 0.22 μm).

PEG (50 mg/mL): 50 g PEG (Sigma Aldrich®, Catalogue ref 94646, 35 kDa) is diluted in a sterile acetic acid solution (500 mM) and made up to 1L. The solution obtained is stirred until a homogeneous and transparent solution is obtained.

PBS 1X: 80 g NaCl (Sigma Aldrich®, 142.04 g/mol); 2 g KCl (Fluka®, 76.5 g/mol); 28.9 g Na2HPO4, 12H2O (Sigma Aldrich®, 358.14 g/mol); 2.027 g NaH2PO4, 1H2O (Sigma Aldrich®, 137.99 g/mol) are diluted in distilled water, then the solution is made up to 1L final volume. Finally, 100 mL of PBS 10X is added to 900 mL of distilled water. The 1L PBS 1X solution is then sterilized.

### Example 1: Preparation of a Transparent Fibrillated Collagen Matrix by Evaporation

Preparation of an Initial Collagen Solution

In this example, an acid-soluble calf collagen solution of 5 mg/mL concentration marketed by SYMATESE under the reference ACI (Baan No. S2132380005), batch number ACI070, is used as initial collagen solution. The pH of the solution is 3.5.

Concentration of the Collagen Solution by Evaporation

100 mL of initial collagen solution in placed in a sterile 200 mL crystallizer previously weighed empty. The crystallizer is left open under a laminar flow hood for approximately 12 hours. Evaporation under the fume hood allows the concentration of the collagen within the container by evaporation of the water present in the solution. A collagen matrix with a concentration of 45 mg/mL is obtained.

The evolution of the collagen concentration during evaporation can be determined by weighing the acid-soluble collagen solution or, more approximately, by measuring the fall in volume within the vessel.

The solution can be regularly homogenized using a pipette during evaporation. If a dry surface film forms, adding several drops of acetic acid allows the mixture to become homogeneous once more and to resume evaporation. A collagen matrix with a collagen concentration of 45 mg/ml is thus obtained.

The same method was reproduced placing 8 ml of the initial collagen solution in a 10 ml crystallizer for 48 hours. A collagen matrix with a concentration of 45 mg/ml is obtained.

Fibrillogenesis of Concentrated Collagen Solutions in the Presence of Ammonia Vapours

The crystallizer containing the collagen solution at a concentration of 45 mg/mL is placed in a desiccator (5L) in which a beaker containing an aqueous ammonia solution (Carlo Erba®, 30 mL, 35.046 g/mol, 28-30%) is also placed. The mixture is left under ammonia vapours in the hermetically sealed desiccator for 2 h under a chemical fume hood. A fibrillated collagen matrix with a pH of 10 is obtained.

Rinsing the Fibrillated Collagen Matrices

Optionally, under the laminar flow hood, the matrix is rinsed with PBS 1X (100 mL, pH=7.4, for 5 minutes) several times in succession until a neutral pH is obtained (pH evaluated with pH paper). The matrix is stored at 4° C. in a sterile Falcon® tube containing PBS 1X (40 mL) until use.

The method described above can be implemented with collagen extracted from rat tails. Transparent matrices of fibrillated collagen have been obtained from an initial collagen solution having an ionic strength of 153 mM, 500 mM, 669 mM or 1.2 M. Similarly, transparent collagen matrices have been obtained from an initial collagen solution with a collagen concentration of 1 mg/mL.

### Example 2: Preparation of a Transparent Fibrillated Collagen Matrix by Dialysis

Preparation of an Initial Collagen Solution

8 mL of a Type I acid-soluble collagen solution (3.2 mg/mL) purified from rat tail tendons according to Gobeaux et al. (Gobeaux et al. 2007. Langmuir, 23 (11), p. 6411-6417) is diluted in 16 mL of Purex sterile acetic acid solution (500 mM). 25 mL of acid-soluble collagen solution with a concentration of 1 mg/mL is obtained. The pH of the solution is 3.5.

Concentration of the Collagen Solution by Dialysis

This collagen solution is injected into a dialysis cell via a Teflon capillary (Dupont® PFA tubing, 0.35 mm internal diameter) using a sterile syringe (Terumo®, 10 mL, needle free). The syringe is pushed using with a syringe driver (KD Scientific®, KDS 101). The dialysis cell is constituted by a mould (QuixSep® Microdialyzers, volume 1 mL) and a dry dialysis membrane rehydrated in boiling water (Spectra/Por®, regenerated cellulose, MWCO 14000 Da).

Dialysis is carried out against a polyethylene glycol solution (PEG, Sigma Aldrich®, 35 kDa, 60 mg/mL, 500 mL) diluted in Purex sterile acetic acid (500 mM). The homogeneity of the solution can be maintained by stirring.

A total volume of 18 mL of collagen solution is injected. During the experiment, the injection speed is set at approximately 0.2 mL/hour, as a function of the “inflation” of the dialysis membrane under the effect of the pressure. With the protocol described above, the injection time for the 18 mL of solution is approximately 3 to 4 hours.

Continuous injection of this solution under dialysis allows the concentration of collagen within the matrix. The form and the size of the matrices depend solely on the mould which constitutes the dialysis cell into which the collagen solution is injected over time. The operating conditions described above make it possible to obtain a matrix of 1 cm³ and of a concentration with a value of 45 mg/mL. A larger mould would involve longer injection times.

All of the assembly is carried out under a laminar flow hood in order to keep the mixture in a sterile condition. The plugs and clips constituting the dialysis cell are previously sterilized by immersion in a beaker containing 90% ethanol (30 minutes). The dialysis membranes are cut to the optimum size (approximately 5 cm in length), then rehydrated by immersion in boiling water then cooled in sterile water.

A sterile syringe (10 mL) is filled with 9 mL of collagen solution. The dialysis cell and the syringe are connected using a capillary, which will allow gradual injection of collagen. In order to make the system hermetic, a sterile cone the end of which is adjusted is connected at one end to the syringe tip wrapped in Teflon, and at the other end to the capillary.
Finally, the dialysis cell is filled with the collagen solution using the syringe (open at the top), then the membrane is placed against the dialysis cell, avoiding the formation of bubbles. The cell is closed using a seal by which it is constituted.
Once the assembly is completed, the tightness of the plug/capillary and capillary/cone junctions is ensured with wax melted using a soldering iron.
The dialysis plugs are placed in a beaker equipped with a bar magnet containing the PEG solution, while ensuring that only the part containing the dialysis membrane is immersed. The beaker is then placed on a magnetic stirrer (400-500 rpm, at ambient temperature) in order to allow homogenization of the PEG concentration through the dialysis membrane.

Fibrillogenesis of the Concentrated Collagen Solution

Once injection of the collagen solution is completed, the injection is stopped. Self-assembly of collagen triple helixes into fibrils occurs by fibrillogenesis under ammonia vapours. To this end, the dialysis cell, without the capillary, is placed in a desiccator (5L) in which a beaker containing an aqueous ammonia solution (Carlo Erba®, 60 mL, 35.046 g/mol, 28-30%) is also placed. The dialysis cell is left under ammonia vapours in the hermetically sealed desiccator for 48 h under a chemical fume hood.

Finally, under the laminar flow hood, the matrix is carefully removed from the dialysis plug using sterile tongs and rinsed several times in a sterile vessel (Corning®, 50 mL, 30×115 mm) containing PBS 1X or sterile ultrapure water (45 mL, for 5 minutes) until a neutral pH is obtained (pH evaluated with pH paper). The matrix is stored at 4° C. in a sterile Falcon® tube containing sterile water (40 mL) until use.

These experimental conditions lead to synthesis of a two-phased fibrillated collagen matrix composed of an opaque phase and a transparent phase superimposed, the latter being removed by cutting using a sterile scalpel and stored.

### Example 3: Characterization of Matrices Obtained

Fibrillated collagen matrices with collagen concentrations of 40 mg/ml, 42 mg/ml, 43.5 mg/mL, 45 mg/mL, 46.8 mg/mL and 48 mg/mL were obtained according to the process disclosed in Example 1 (calf collagen).

The file of this patent contains at least one drawing executed in color. Copies of this patent with color drawings will be provided by the Patent and Trademark Office upon request and payment of the necessary fee.”

Observations with the Naked Eye and with Polarized Light Microscopy (PLM)

FIG. 1 summarizes the results of analysis with the naked eye and with polarized light microscopy of collagen matrices with different concentrations comprised between 40 mg/ml and 48 mg/ml of collagen.

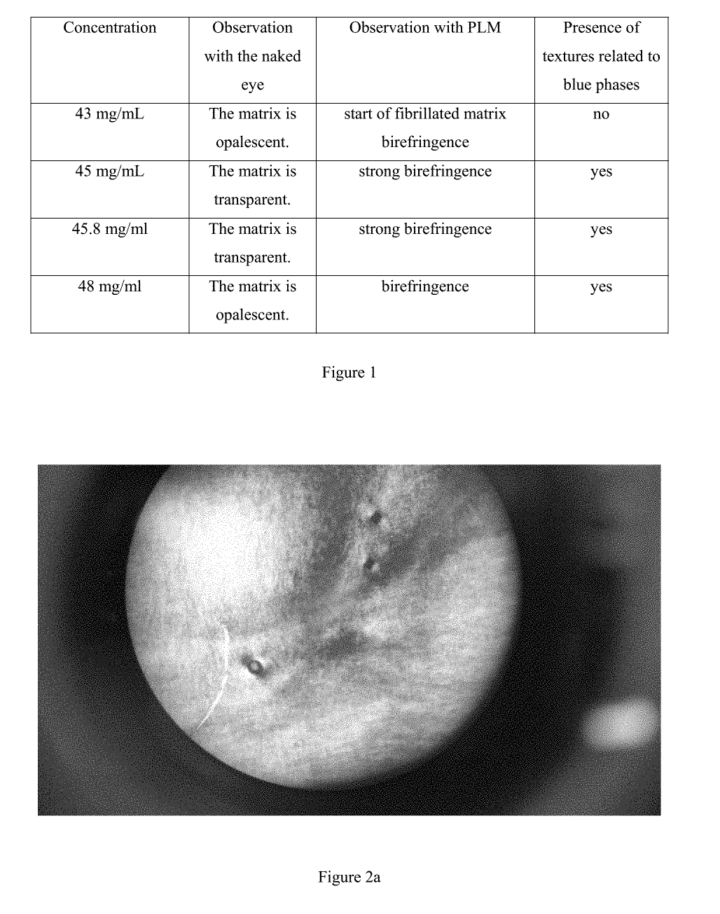

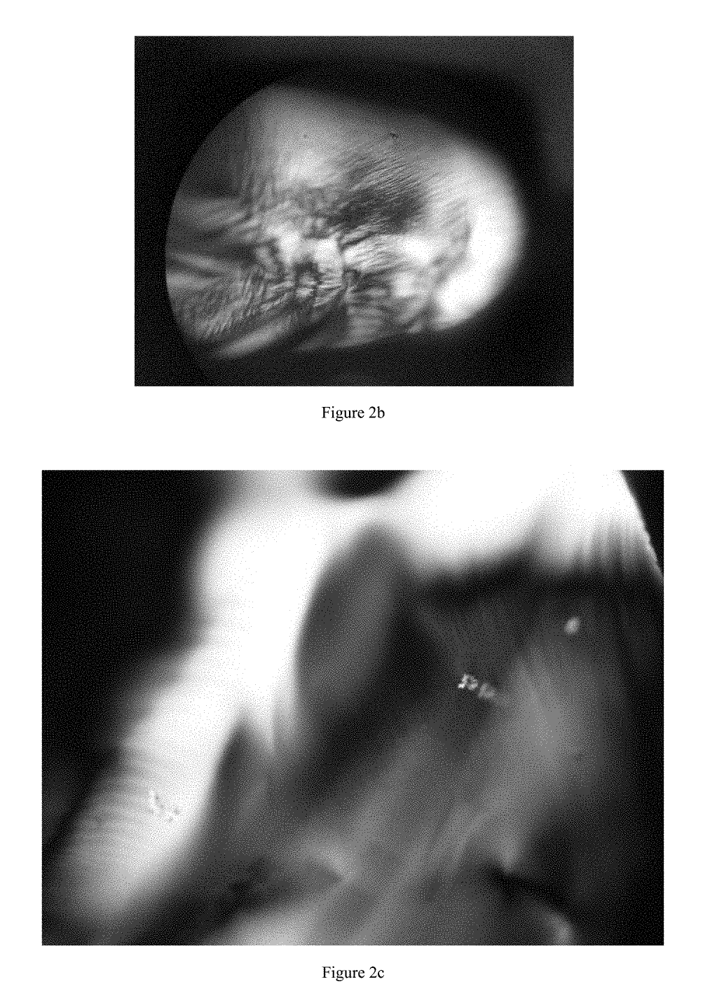

FIG. 2a shows the presence of birefringent organizations related to “blue phases” in a collagen solution concentrated at 45 mg/mL. These particular crystalline-liquid phases have not been described until now, except for thermotropic liquid crystals. In solution, collagen is a liquid crystal of the lyotropic type. Observation of such a “blue phase” is consistent to the extent that it is described in the literature, for thermotropic liquid crystals, as being an intermediate phase between the isotropic phase and the cholesteric phase.

FIG. 2b shows production of a comparable texture in a solution of collagen from rat tails concentrated at 45 mg/mL.

FIG. 2c shows the presence of birefringent organizations related to “blue phases” in a fibrillated collagen matrix concentrated at 45 mg/mL, thus indicating that this particular organization was stored after fibrillogenesis.

Study of Transmittance by Ellipsometry

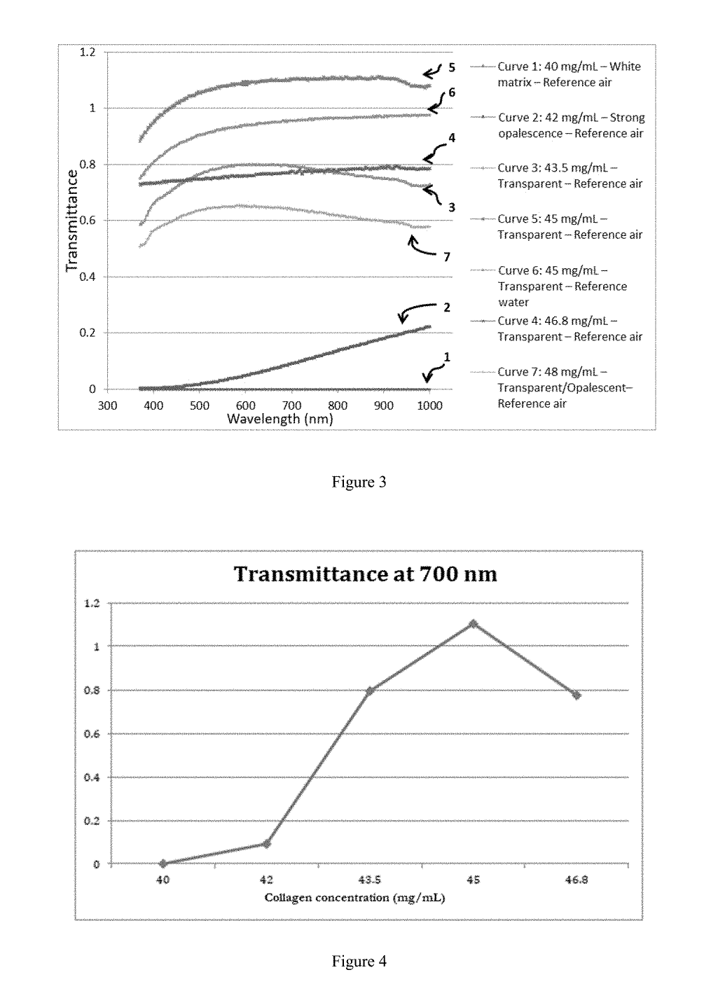

The transmittance of fibrillated collagen matrices has been studied by ellipsometry (FIGS. 3 and 4). The transmission spectra are obtained using an Ellipsometer (Variable angle spectroscopic ellipsometry (VASE) M-2000U Woollam spectroscopic ellipsometer) between 370 and 1000 mm at an angle of incidence of 0°. In order to ensure the planarity of the surfaces, the matrices are placed between two glass slides for microscopy using air for reference (Faustini et al. ACS Appl. Mater. Interfaces, 2014, 6 (19), pp 17102-17110).

FIG. 3 shows the transmittance of the matrices between 370 and 1000 nm. The transmittance of the matrices of concentrations is shown on the Y-axis as a function, on the X-axis, of the wavelength in nm: 40 mg/ml (curve 1), 42 mg/ml (curve 2), 43.5 mg/mL (curve 3), 45 mg/mL (curve 5), 46.8 mg/mL (curve 4) and 48 mg/ml (curve 7) as a function of the wavelength (370 to 1000 nm). The references are air. For the 45 mg/mL matrix, water was also used for reference (curve 6). Increased transmittance regardless of the wavelength is obtained with the 45 mg/mL matrix.

FIG. 4 shows the transmittance of the matrices at 700 nm. It can be seen that the transmittance reaches a maximum for fibrillated collagen matrices with a concentration of 45 mg/ml and that moving away from the value of 45 mg/mL, transmittance decreases. The matrices with a concentration of 40 mg/ml are opaque.

### Example 4: Effect of a Shearing Stress on a Matrix Before Fibrillogenesis

FIG. 5 shows the importance of limiting the shearing stresses before fibrillogenesis. Area 1 shows an area of a fibrillated collagen matrix with a collagen concentration of 45 mg/ml having only been subjected to shearing stresses associated with manipulations during the concentration step. Area 1 of the collagen matrix, transparent before fibrillogenesis, remains transparent after fibrillogenesis. Area 2 shows an area of a fibrillated collagen matrix with a collagen concentration of 45 mg/ml having been subjected to the application of a local mechanical stress in the form of surface friction using a scalpel before fibrillogenesis. Area 2 of this collagen matrix, transparent before fibrillogenesis, becomes opaque after fibrillogenesis. Area 3 of this collagen matrix shows an area of a fibrillated collagen matrix with a collagen concentration of 45 mg/ml having been subjected to sampling using a spatula having led to a local mechanical stress before fibrillogenesis. It can be seen that only the edges of this Area 3 of this matrix have become opaque. Manipulation of the matrix after fibrillogenesis does not lead to the appearance of areas of opaqueness.

### Example 4: Storing the Matrices

Matrices obtained by the method according to the invention from collagen from rat tails have been stored for 4 years at 4° C. in sterile ultrapure water with no alteration in transparency.

The inventors have also stored matrices obtained by the method of the invention from clinical grade collagen in PBS at 4° C. supplemented with a storage medium intended for corneas (Cornea cold®, Eurobio, Les Ulis, France) for 3 weeks with no alteration in transparency.

A film of salts can form on the surface when the matrices are stored in cold PBS in the case of “clinical grade” collagen, but it is possible for it to be removed. Precipitation of salts can be avoided and storage times increased in a storage medium intended for corneas (Cornea cold®) remaining at 4° C.

### REFERENCES

Hulmes D. J. S., Miller A. 1979. Nature, 282, p. 878-880.
Hélary et al. 2005. Biomaterials, 26, p. 1533-1543
Gobeaux et al. 2007. Langmuir, 23 (11), p. 6411-6417
Knight et al. 1998. J Biomed Mater Res. 41, p. 185-191
Faustini et al. 2014. ACS Appl. Mater. Interfaces, 6 (19), p. 17102-17110

1. Method for preparing a transparent matrix of fibrillated collagen comprising the following steps:
a) a step of preparing a collagen acid solution in an aqueous solvent
b) a step of concentrating the collagen acid solution
c) a step of fibrillogenesis of the collagen matrix wherein
the concentration step is carried out until a collagen concentration is obtained comprised between 43 and 50 mg/ml.

2. Method according to claim 1, wherein the concentration step is carried out so as to limit, advantageously to avoid, shearing stresses.

3. Method according to claim 1, wherein the concentration step is carried out until a collagen concentration is obtained comprised between 43.5 and 47 mg/mL, preferentially of the order of 45 mg/mL.

4. Method according to claim 1, wherein the collagen acid solution is constituted by an aqueous acetic acid solution.

5. Method according to claim 3, wherein the concentration of the initial acid solution, before the concentration step, is comprised between 0.01 mg/ml and 5 mg/mL, advantageously comprised between 0.5 and 3 mg/mL.

6. Method according to claim 1, wherein the concentration step is carried out by evaporation or by dialysis.

7. Method according to claim 1, wherein the fibrillogenesis step consists of bringing the collagen matrix into contact with a basic gas phase or a neutral or basic liquid.

8. Method according to claim 7, wherein the fibrillogenesis step is carried out by bringing the collagen matrix into contact with ammonia vapours.

9. Method according to claim 1, also comprising a step of washing the fibrillated collagen matrix in a buffer solution, for example phosphate buffered solution.

10. Method for preparing a transparent collagen matrix according to claim 1, further comprising the following steps:
a) preparing a collagen acid solution in an aqueous acetic acid solution at a collagen concentration comprised between 0.1 and 5 mg/mL, advantageously between 0.2 and 1 mg/mL;
b) concentrating the collagen acid solution by evaporation or by dialysis until a collagen concentration is obtained comprised between 42 and 50 mg/mL;
c) forming fibrils by bringing the collagen matrix into contact with a basic gas phase or a neutral or basic liquid, advantageously by bringing the collagen matrix into contact with ammonia vapours.

11. Transparent fibrillated collagen matrix capable of being obtained by a method according to claim 1, wherein the matrix has a transmittance greater than 0.6 at 700 nm.

12. A tissue or an artificial organ comprising the fibrillated collagen matrix of claim 11.

13. A dressing comprising the fibrillated collagen matrix according to claim 11.

14. Method according to claim 2, also comprising a step of washing the fibrillated collagen matrix in a buffer solution, for example phosphate buffered solution.

15. Method according to claim 3, also comprising a step of washing the fibrillated collagen matrix in a buffer solution, for example phosphate buffered solution.

16. Method according to claim 4, also comprising a step of washing the fibrillated collagen matrix in a buffer solution, for example phosphate buffered solution.

17. The tissue of claim 12, wherein the tissue is a cornea substitute.

18. The transparent fibrillated collagen matrix of claim 11, wherein the matrix has a transmittance greater than 0.8 at 700 nm.

19. The method of claim 10, wherein in further step b) the obtained collagen concentration is of the order of 45 mg/mL.

20. The method of claim 10, wherein in further step b) the obtained collagen concentration is between 43 and 47 mg/mL.
